(19) United States
(12) Patent Application Publication
(10) Pub. No.: US 2005/0260609 A1
(43) Pub. Date: Nov. 24, 2005

(54) Methods and devices for sequencing nucleic acids

(76) Inventor: Stanley N. Lapidus, Bedford, NH (US)

Correspondence Address: PROSKAUER ROSE LLP, ONE INTERNATIONAL PLACE 14TH FL, BOSTON, MA, 02110, US

(21) Appl. No.: 10/852,028

(22) Filed: May 24, 2004

(51) Int. Cl. C12Q 1/68; C07H 21/04

(52) U.S. Cl. 435/6; 536/24.3

(57) ABSTRACT

The invention provides methods and devices for high throughput single molecule sequencing of a plurality of target nucleic acids using a universal primer. Devices of the invention comprise a plurality of oligonucleotides, each having the same sequence, bound to a solid support, and ligated to a plurality of target nucleic acids.

### FIELD OF THE INVENTION

The invention relates to methods and devices for sequencing a nucleic acid, and more particularly, to methods and devices for high throughput single molecule sequencing of target nucleic acids.

### BACKGROUND

Completion of the human genome has paved the way for important insights into biologic structure and function. Knowledge of the human genome has given rise to inquiry into individual differences, as well as differences within an individual, as the basis for differences in biological function and dysfunction. For example, single nucleotide differences between individuals, called single nucleotide polymorphisms (SNPs), are responsible for dramatic phenotypic differences. Those differences can be outward expressions of phenotype or can involve the likelihood that an individual will get a specific disease or how that individual will respond to treatment. Moreover, subtle genomic changes have been shown to be responsible for the manifestation of genetic diseases, such as cancer. A true understanding of the complexities in either normal or abnormal function will require large amounts of specific sequence information.

An understanding of cancer also requires an understanding of genomic sequence complexity. Cancer is a disease that is rooted in heterogeneous genomic instability. Most cancers develop from a series of genomic changes, some subtle and some significant, that occur in a small subpopulation of cells. Knowledge of the sequence variations that lead to cancer will lead to an understanding of the etiology of the disease, as well as ways to treat and prevent it. An essential first step in understanding genomic complexity is the ability to perform high-resolution sequencing. Bulk sequencing techniques simply do not have the resolution necessary to detect the subtle and specific changes that underlie cancer.

One conventional way to do bulk sequencing is by chain termination and gel separation, essentially as described by Sanger et al., Proc Natl Acad Sci USA, 74(12): 5463-67 (1977). That method relies on the generation of a mixed population of nucleic acid fragments representing terminations at each base in a sequence. The fragments are then run on an electrophoretic gel and the sequence is revealed by the order of fragments in the gel. Another conventional bulk sequencing method relies on chemical degradation of nucleic acid fragments. See, Maxam et al., Proc. Natl. Acad. Sci., 74: 560-564 (1977). Finally, methods have been developed based upon sequencing by hybridization. See, e.g., Drmanac, et al., Nature Biotech., 16: 54-58 (1998).

Recent developments in sequencing technology include methods in which the target nucleic acids are attached to a solid surface and incubated in the presence of a polymerase and nucleotide analogues that have a blocker at the 3′ hydroxyl. An incorporated analog is detected. Following detection, the blocking group is cleaved, typically, by photochemical means to expose a free hydroxyl group that is available for base addition during the next cycle.

Techniques utilizing 3′ blocking are prone to errors and inefficiencies. For example, those methods require excessive reagents, including numerous primers complementary to at least a portion of the target nucleic acids and differentially-labeled nucleotide analogues. They also require additional steps, such as cleaving the blocking group and differentiating between the various nucleotide analogues incorporated into the primer. As such, those methods have only limited usefulness.

A need therefore exists for more effective and efficient methods and devices for single molecule nucleic acid sequencing.

### SUMMARY OF THE INVENTION

The invention provides methods and devices for sequencing nucleic acids. In particular, the invention provides a substrate comprising a plurality of oligonucleotides, each having the same sequence, for use as a platform for high throughput single molecule sequencing using a universal primer.

In general terms, the invention provides a solid support and a plurality of oligonucleotides, each having the same sequence. The oligonucleotides are attached to the solid support in a spatial arrangement that allows all or some of them to be individually optically resolvable. Oligonucleotides of the invention are of any sequence length that is capable of hybridizing to a primer for template-dependent synthesis. Typical oligonucleotides for use in the invention comprise between at least about 5 and about 100 nucleotides. Oligonucleotides of the invention further comprise a primer attachment site and a terminal attachment site for attaching a target polynucleotide. Oligonucleotides of the invention may be oligodeoxynucleotides or oligodeoxyribonucleotides, and may include, in whole or in part, non-naturally occurring nucleotides or modified nucleotides. For example, oligonucleotide sequences may contain peptide nucleic acids (PNAs) or other analogs. Oligonucleotides may also comprise a detectable label in some embodiments.

According to the invention, a plurality of target polynucleotides are attached to the support-bound oligonucleotides described above, one target polynucleotide per oligonucleotide, in order to produce a plurality of chimeric polynucleotides arrayed on the substrate. Target polynucleotides are attached to the oliogonucleotides through any convenient mode of attachment, such as blunt-end or cohesive-end ligation, or others known in the art. Oligonucleotides are attached to the solid support either before or after attachment to target polynucleotides. For example, oligonucleotides and target polynucleotides may be ligated together in solution, then attached to a solid support. Alternatively, oligonucleotides may first be attached to the solid support and then ligated to target polynucleotides. Target polynucleotides typically, although not necessarily, are longer than oligonucleotides. Preferred targets comprise nucleic acid obtained from a biological sample. The targets may be isolated and prepared prior to attachment to the oligonucleotides, or may be exposed as a crude preparation of nucleic acid and other cellular material.

Accordingly, the invention provides a universal array of oligonucleotides that is useful for sequencing any target polynucleotide. The fact that the oligonucleotides are identical allows the use of a universal primer in a sequencing-by-synthesis reaction to determine a sequence of an attached polynucleotide target.

The surface to which oligonucleotides are attached may be chemically modified to promote attachment, improve spatial resolution, and/or reduce background. Exemplary substrate coatings include polyelectrolyte multilayers. Typically, these are made via alternate coatings with positive charge (e.g., polyllylamine) and negative charge (e.g., polyacrylic acid). Alternatively, the surface can be covalently modified, as with vapor phase coatings using 3-aminopropyltrimethoxysilane. Oligonucleotides may be attached to the surface by a chemical linkage, such as a biotin/streptavidin, digoxigenin/anti-digoxigenin, or others known in the art. Typical supports for use in the invention include glass or fused silica slides. However, the invention also contemplates the use of beads or other non-fixed surfaces. Solid supports of the invention may comprise glass, plastic, metal, nylon, gel matrix or composites. According to the invention, oligonucleotides are arranged on the solid surface by, for example, microfluidic spotting techniques or patterned photolithography, in a spatial relationship such that each of the oligonucleotide is individually optically resolvable (i.e., can be distinguished optically from other oligos in the array). For example, the oligonucleotides may be bound to the solid support at precisely defined locations at a density sufficiently low to permit each of the oligonucleotides to be individually optically resolvable. Substrates of the invention may comprise at least about 50, 100, 200, 500, 1000, 2500, 5000, 10,000, 20,000 or 50,000 different oligonucleotides, each being available for attachment to a target polynucleotide.

Generally, in use, a substrate comprising a plurality of chimeric polynucleotides (i.e., individual oliogonucleotides attached to a target polynucleotide as described herein) is exposed to a plurality of primers, each having the same sequence and being capable of hybridizing to a primer attachment site on the oligonucleotide portion of the chimeric structure. The primer is extended in the presence of one or more nucleotides comprising a detectable label. Incorporation of label, if any, is then determined for all or a subset of the chimeric polynucleotides.

Alternatively, a substrate comprising a plurality of primers, each having the same sequence and being capable of hybridizing to the primer attachment site of the oligonucleotides, is prepared. The substrate is exposed to a plurality of chimeric polynucleotides and the primer is extended in the presence of one or more nucleotides comprising a detectable label. The incorporation of the label is then determined for each of the chimeric polynucleotides. Thus, the primers may be anchored to the substrate and serve to capture oligonucleotides by hybridization.

Labeled nucleotides for use in the invention are any nucleotide that has been modified to include a label that is directly or indirectly detectable. Preferred labels include optically-detectable labels, including fluorescent labels, such as fluorescein, rhodamine, derivatized rhodamine dyes, such as TAMRA, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, 120 ALEXA, or a derivative or modification of any of the foregoing. As the skilled artisan will appreciate, however, any detectable label can be used to advantage within the principles of the invention.

While the invention is useful to detect single nucleotides (i.e., to perform single base extensions), the steps of extending the chimeric polynucleotides and detecting incorporated label are repeated in order to generate multibase sequences. For example, the universal primer is extended in the presence of a single species of a nucleotide comprising a detectable label, the incorporation of which is then determined. The primer is then extended in the presence of a different single species of labeled nucleotide, the incorporation of which is determined. By repeating these steps, a sequence of the attached target polynucleotide is determined as the complement of the extended primer sequence. In order to decrease background caused by previously incorporated labeled nucleotides, the invention further provides as an alternative that once detected, an incorporated label is silenced by quenching, photobleaching, cleavage or any other mode of abating or eliminating the detectable signal produced by the label. Labeled nucleotides for use in the invention may also be nucleotide analogs, such as peptide nucleic acids, acyclonucleotides, and others known in the art.

In one embodiment, methods of the invention comprise fluorescence resonance energy transfer (FRET) as a convenient way to detect incorporation of nucleotides in the extending primer strand. Fluorescence resonance energy transfer in the context of sequencing is described generally in Braslavasky, et al., Proc. Nat'l Acad. Sci., 100: 3960-3964 (2003), incorporated by reference herein. Essentially, a donor fluorophore is attached to the primer (or in some cases to polymerase). Nucleotides added for incorporation into the primer comprise an acceptor fluorophore that can be activated by the donor when the two are in proximity. Activation of the acceptor causes it to emit a characteristic wavelength of light and also quenches the donor. In this way, incorporation of a nucleotide in the primer sequence is detected by detection of acceptor emission.

Preferred methods of the invention are directed to detection of single nucleic acid molecules using fluorescent microscopy. Thus, according to the invention, single nucleotide incorporations are imaged as a complement strand is synthesized by polymerase. After each successful incorporation, a fluorescent signal is observed and then nullified. Fluorescent observation is accomplished using conventional microscopy as described below. The invention allows the observation of successive incorporations into individual nucleic acid complement molecules. This provides a significant advantage over bulk detection methods that do no allow single molecule resolution. For example, methods of the invention allow detection of a single nucleotide difference in a small subpopulation of template molecules in a sample. Moreover, the invention allows the resolution of single molecule differences across individuals or within individuals. Single molecule resolution also allows one to determine expression patterns, active splice variants, and other aspects of nucleic acid function.

The invention also provides substrates for the analysis of nucleic acid samples. In a preferred embodiment, a substrate of the invention comprises a plurality of oligonucleotides, each having the same sequence. The oligonucleotides may be covalently bound to the substrate or they may be attached by more transient means. A preferred substrate of the invention further comprises primer that is capable of attaching to a primer binding site present on each of the oligonucleotides. One embodiment of the invention is a kit comprising a substrate having a plurality of same-sequence oligonucleotides bound to a substrate surface, a primer capable of hybridizing with a primer attachment site on each of the oligonucleotides, a polymerase capable of catalyzing template-specific nucleotide addition to the primer, and an appropriate buffer. In other embodiments, the kit contains buffer, enzymes, and other factors known in the art to promote ligation of a target to the bound oligonucleotides. The specific buffers and enzymes, as well as reaction conditions, are determined at the convenience of the user, and are based upon well-known factors specific to the sequences being used. Preferred polymerases include Klenow, TAQ, Vent, Terminator, Nine Degrees North, Keno, all preferably lacking exonuclease activity. In practice, a sample containing target polynucleotide to be sequenced is applied to substrate and ligated to the oligonucleotides bound thereto in order to form chimeric polynucleotides. The kit is then exposed to polymerase, buffer and labeled nucleotides in succession in order to construct complement to the chimeric sequences. Added nucleotides are observed based upon their optical signals as described herein, and a sequence is compiled by appropriate software.

A detailed description of the certain embodiments of the invention is provided below. Other embodiments of the invention are apparent upon review of the detailed description that follows.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows an embodiment of a substrate of the invention including a solid support and chimeric polynucleotides attached thereto.

FIG. 2 is a diagrammatic representation of an exemplary method of the invention.

FIG. 3 is a screen shot showing inputs used in a model of stochastic base addition in a single molecule sequencing by synthesis reaction.

FIG. 4 is a series of screenshots showing the effects of altering reaction conditions on the incorporation of nucleotides in a single molecule sequencing by synthesis reaction.

FIG. 5 is a diagram of a FRET-based single molecule nucleotide addition.

### DETAILED DESCRIPTION

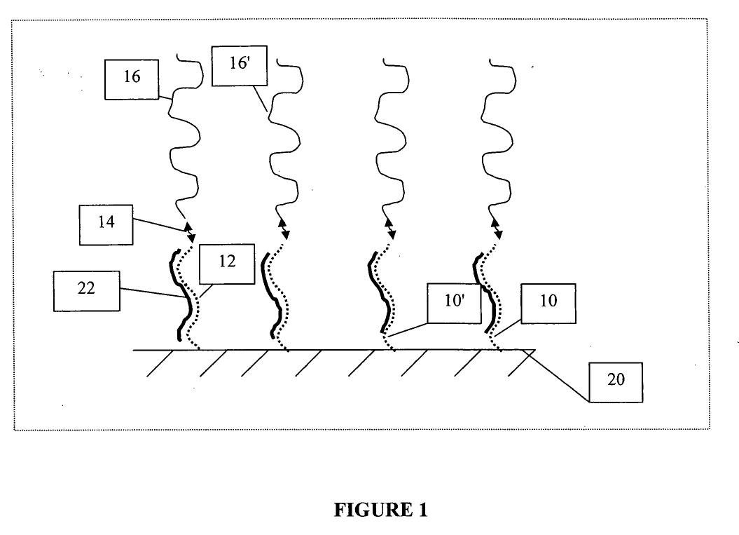

The invention provides methods and devices for high throughput single molecule sequencing of target nucleic acids using a universal primer. As shown in FIG. 1, at its most basic level, the invention provides a plurality of oligonucleotides (10, 10′), each having the same sequence comprising both a primer attachment site (12) and a terminal attachment site (14) for a target nucleic acid. Each of the target nucleic acids (16, 16′) is attached to an oligonucleotide (10, 10′), producing a chimeric polynucleotide. Either before or after the target nucleic acids (16, 16′) are attached to the oligonucleotides, the oligonucleotides are bound to a solid support (20) in a spatial arrangement such that each individual oligonucleotide (10, 10′) is optically-resolvable. Because each target nucleic acid (16, 16′) is attached to an oligonucleotide (10, 10′) comprising the same sequence (and thus the same primer attachment site (12)), a single universal primer (22) can be employed in single molecule sequencing techniques comprising base extensions, such as those described in Braslavky et al. (2003) PNAS 100(7), 3960-64 (incorporated by reference herein), or any technique involving the synthesis of a plurality of nucleic acid that are complementary to the target nucleic acids.

Methods and devices of the invention are useful for analyzing nucleic acids of any type and from any source, such as animal, plant, bacteria, virus, fungus, or synthetically made. For example, target nucleic acids may be naturally occurring DNA or RNA, recombinant molecules, genomic DNA, cDNA or synthetic analogs (e.g., PNAs and others). Further, target nucleic acids may be a specific portion of a genome of a cell, such as an intron, regulatory region, allele, variant or mutation; the whole genome; or any portion between. In other embodiments, the target nucleic acids may be mRNA, tRNA, rRNA, ribozymes, antisense RNA or siRNA. The target nucleic acid may be of any length, such as at least about 10, 25, 50, 100, 500, 1000, or 2500 bases. While the target nucleic acid may be amplified by, for example, polymerase chain reaction, prior to sequencing, it need not be.

Additional aspects of the invention are described in the following sections and illustrated by the Examples.

Substrates

Typical solid supports of the invention comprise a planar surface, such as a glass or fused silica slide. However, the invention also provides for three-dimensional solid supports, such as beads and the like. A solid support of the invention may comprise glass, quartz, plastic (such as polystyrene, polycarbonate, polypropylene and poly(methymethacrylate)), metal, nylon, gel matrix or composites. In a preferred embodiment, the solid support comprises a biocompatible or biologically inert material that is transparent to light and optically flat (i.e., with a minimal microroughness rating).

Typical three-dimensional solid supports includes microarray reaction chambers, but three-dimensional solid supports may take the form of, for example, spheres, tubes (e.g., capillary tubes), microwells, microfluidic devices, or any other form suitable for supporting the oligonucleotides.

In some embodiments, the solid supports are associated or chemically modified with one or more coatings or films that increase the oligonucleotide-to-support binding affinity, reduce background, and/or improve positioning of the bound oligonucleotides or chimeric polynucleotides. Increased oligonucleotide binding to substrates leads to increased retention of the oligonucleotides and chimeric polynucleotides during the various stages of substrate preparation and analysis (e.g., hybridization, primer extension, washing, label detection, label abatement, etc). Exemplary coatings include avidin or streptavidin (when used as a linker with biotin), and vapor phase coatings of 3-aminopropyltrimethoxysilane. In a preferred embodiment, the solid support surface is a polyelectrolyte multilayer formed by alternate treatment with polyllylamine and polyacrylic acid. The carboxyl groups of the polyacrylic acid layer are negatively charged and thus repel negatively charged labeled nucleotide, improving the positioning of the label for detection.

Support coatings are also made to reduce background emission. For example, polyethylene compounds, such as polytetrafluorethylene, that typical repel background particulate matter are useful.

Oligonucleotides and Primers

Any oligonucleotide sequence is useful in the invention as long as each substrate for use in the invention contains oligonucleotides of the same sequence. Oligonucleotides of any length capable of forming chimerics and supporting polymerase-directed, template-dependent sequencing are useful. Typically, oligonucleotides comprise from about at least 5 to about 100 nucleotides, and include a primer attachment site and a terminal attachment site for attaching a target nucleic acid. Oligonucleotides of the invention may be oligodeoxynucleotides or oligodeoxyribonucleotides, and may include, in whole or in part, modified or non-naturally occurring nucleotides, including, for example a peptide nucleotide. Furthermore, oligonucleotides of the invention may comprise modified phosphate-sugar backbones.

Primers useful in the invention comprise a sequence complementary to the primer attachment site of whatever oligonucleotide sequence is being used. While the primers may hybridize solely with the primer attachment site of the oligonucleotides, primers may also span beyond the 3′ end of the oligonucleotide to hybridize with a 5′ portion of the target nucleic acid as well. Depending on the oligonucleotide used, the primer may be DNA, RNA or a mixture of both. According to one embodiment of the invention, the primers comprise at least 5, 10, 15, 20, 30, 40 or 50 nucleotides.

Oligonucleotides and primers of the invention can be made synthetically using conventional nucleic acid synthesis technology. For example, the oligonucleotides and primers can be synthesized via standard phosphoramidite technology utilizing a nucleic acid synthesizer. Such synthesizers are available, e.g., from Applied Biosystems, Inc. (Foster City, Calif.). Alternatively, the oligonucleotides and primers can be purchased commercially from companies such as Operon Inc. (Alameda, Calif.).

In the event that the oligonucleotides are to be attached to the solid support prior to ligation with the target nucleic acids, the oligonucleotides can be synthesized in situ using, for example, soft lithography or photolithography techniques.

Ligation of the Oligonucleotides to the Target Nucleic Acids

According to the invention, a plurality of target nucleic acids are attached at the terminal attachment site of the oligonucleotides, one target nucleic acid per oligonucleotide, thereby producing a plurality of chimeric polynucleotides. The target nucleic acids may be attached to the oligonucleotides either before or after the oligonucleotides are attached to the solid support. The target nucleic acids are attached to the oligonucleotides through any mode of attachment that results in the creation of a phosphodiester bond between the 5′ phosphate of the target nucleic acid nucleotide and the 3′ hydroxyl of the oligonucleotide. The oligonucleotides and target nucleic acids may be ligated in a single-stranded form, or a double-stranded form by either blunt-end or cohesive-end ligation. Ligases useful in the invention include, for example T4 DNA ligase, E. coli ligase and Ampligase DNA ligase. In one embodiment, double-stranded chimeric polynucleotides are reduced to single strands by, for example, subjecting the double-stranded polynucleotides to a temperature that causes destabilization of the hydrogen bonds between the strands, or by subjecting the polynucleotides to a low salt solution.

Attachment of the Oligonucleotides to the Solid Support

According to the invention, oligonucleotides are attached to the solid support either before or after the target nucleic acids are attached to the oligonucleotides. Alternatively, primers are attached to the solid support by any method useful in attaching an oligonucleotide. In one embodiment, the oligonucleotides are attached to the solid support directly by cross-linking to an unmodified surface by conjugating an active silyl moiety onto the oligonucleotide. Alternatively, oligonucleotides may be attached to the solid support via a linker group. Ideally, the linker group does not significantly interfere with either the primer binding to the oligonucleotide or the activity of polymerase. The linker can be a covalent or non-covalent mode of attachment. In one embodiment, the linker comprises a pair of molecules having a high affinity for one another, one molecule on the oligonucleotide and the other on the solid support. Such pairs include biotin and avidin, histidine and nickel, digoxigenin and anti-digoxigenin, and GST and glutathione.

Other linkers useful in attaching the oligonucleotide to the solid support include straight-chain or branched amino- or mercapto-hydrocarbon with more than two carbon atoms in the unbranched chain, such as aminoalkyl and aminoalkynyl groups. Alternatively, the linker may be any alkyl chain of 10-20 carbons in length, and may be attached through an Si—C direct bond or through an ester Si—O—C linkage.

According to the invention, oligonucleotides are arranged on the solid support by microfluidic spotting techniques, patterned photolithographic synthesis, or ink-jet printing, or any other method in a spatial relationship such that each of the oligonucleotide is optically resolvable. The oligonucleotides may be bound to the solid support at precisely defined locations on a solid support, or may be bound randomly at a sufficiently low such that each oligonucleotide is optically resolvable. Substrates of the invention may comprise at least about 50, 100, 200, 500, 1000, 2500, 5000 or 10,000 chimeric polynucleotides.

Incorporation of Labeled Nucleotides

Generally, in use, a substrate comprising a plurality of chimeric polynucleotides (i.e., individual oligonucleotides, each attached to a target nucleic acid) is exposed to a plurality of primers, each having the same sequence and capable of hybridizing to the primer attachment site of the oligonucleotides. The primer is extended in the presence of one or more nucleotides comprising a detectable label. The incorporation of the label is then determined. This experiment is repeated, sequentially alternating the species of labeled nucleotide, such that a sequence is compiled from which the sequence of the target nucleic acid can be determined.

Labeled nucleotides of the invention include any nucleotide that has been modified to include a label that is directly or indirectly detectable. Such labels include optically-detectable labels such as fluorescent labels, including fluorescein, rhodamine, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, ALEXA, TAMRA, or a derivative or modification of any of the foregoing. In one embodiment of the invention, fluorescence resonance energy transfer (FRET) is employed to produce a detectable, but quenchable, label. FRET may be used in the invention by, for example, modifying the primer to include a FRET donor moiety and using nucleotides labeled with a FRET acceptor moiety.

While the invention is exemplified herein with fluorescent labels, the invention is not so limited and can be practiced using nucleotides labeled with any form of detectable label, including radioactive labels, chemoluminescent labels, luminescent labels, phosphorescent labels, fluorescence polarization labels, and charge labels.

### EXAMPLES

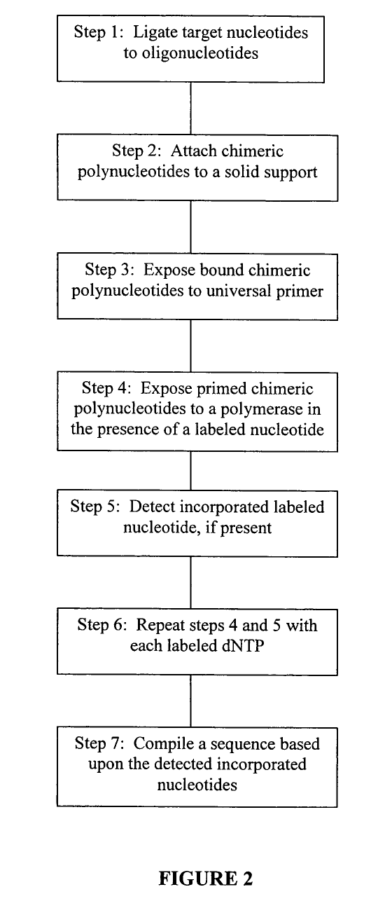

In this example, target nucleic acids are ligated to an oligonucleotide and bound to a solid support. The chimeric polynucleotides are exposed to a universal primer in the presence of a labeled nucleotide. If the labeled nucleotide is incorporated into the primer, the label is detected and recorded. By repeating the experimental protocol with each of labeled dCTP, dUTP, dATP, and dGTP, a sequence is compiled that is representative of the complement of the target nucleic acid. This process is depicted diagrammatically in FIG. 2.

Oligonucleotide and Primer Preparation

For this experiment, an oligonucleotide is designed to meet the following criteria: (a) the oligonucleotide must contain a primer attachment site that allows for specific hybridization of a primer; (b) the oligonucleotide must permit ligation with a target nucleic acid; (c) the oligonucleotide must permit attachment to a solid support; and (d) the tertiary structure of the oligonucleotide must permit primer attachment, polymerase activity and signal detection. For the purpose of this example, the oligonucleotide is designed that comprises a 25-mer primer attachment site having a high G-C content to provide a more stable duplex with the primer, a free 3′ hydroxyl group and a 5′ biotinylated terminus. The universal primer is designed as 25-mer complementary to the primer attachment site of the oligonucleotide, and comprises a Cy3 tag at the 5′ terminus.

The oligonucleotides and primers are synthesized from nucleoside triphosphates by known automated oligonucleotide synthetic techniques, e.g., via standard phosphoramidite technology utilizing a nucleic acid synthesizer, such as the ABI3700 (Applied Biosystems, Foster City, Calif.). The oligonucleotides are prepared as duplexes with a complementary strand, however, only the 5′ terminus of the oligonucleotide proper (and not its complement) is biotinylated.

Ligation of Oligonucleotides and Target Polynucleotides

Double stranded target nucleic acids are blunt-end ligated to the oligonucleotides in solution using, for example, T4 ligase. The single strand having a 5′ biotinylated terminus of the oligonucleotide duplex permits the blunt-end ligation on only one end of the duplex. In a preferred embodiment, the solution-phase reaction is performed in the presence of an excess amount of oligonucleotide to prohibit the formation of concantamers and circular ligation products of the target nucleic acids. Upon ligation, a plurality of chimeric polynucleotide duplexes result. Chimeric polynucleotides are separated from unbound oligonucleotides based upon size and reduced to single strands by subjecting them to a temperature that destabilizes the hydrogen bonds.

Preparation of Solid Support

A solid support comprising reaction chambers having a fused silica surface is sonicated in 2% MICRO-90 soap (Cole-Parmer, Vernon Hills, Ill.) for 20 minutes and then cleaned by immersion in boiling RCA solution (6:4:1 high-purity H₂O/30% NH₄OH/30% H₂O₂) for 1 hour. It is then immersed alternately in polyallylamine (positively charged) and polyacrylic acid (negatively charged; both from Aldrich) at 2 mg/ml and pH 8 for 10 minutes each and washed intensively with distilled water in between. The slides are incubated with 5 mM biotin-amine reagent (Biotin-EZ-Link, Pierce) for 10 minutes in the presence of 1-[3-(dimethylamino)propyl]-3-ethylcarbodiimide hydrochloride (EDC, Sigma) in MES buffer, followed by incubation with Streptavidin Plus (Prozyme, San Leandro, Calif.) at 0.1 mg/ml for 15 minutes in Tris buffer. The biotinylated single-stranded chimeric polynucleotides are deposited via ink-jet printing onto the streptavidin-coated chamber surface at 10 pM for 10 minutes in Tris buffer that contain 100 mM MgCl₂.

Equipment

The experiments are performed on an upright microscope (BH-2, Olympus, Melville, N.Y.) equipped with total internal reflection (TIR) illumination, such as the BH-2 microscope from Olympus (Melville, N.Y.). Two laser beams, 635 (Coherent, Santa Clara, Calif.) and 532 nm (Brimrose, Baltimore), with nominal powers of 8 and 10 mW, respectively, are circularly polarized by quarter-wave plates and undergo TIR in a dove prism (Edmund Scientific, Barrington, N.J.). The prism is optically coupled to the fused silica bottom (Esco, Oak Ridge, N.J.) of the reaction chambers so that evanescent waves illuminated up to 150 nm above the surface of the fused silica. An objective (DPlanApo, 100 UV 1.3oil, Olympus) collects the fluorescence signal through the top plastic cover of the chamber, which is deflected by the objective to ˜40 μm from the silica surface. An image splitter (Optical Insights, Santa Fe, N. Mex.) directs the light through two bandpass filters (630dcxr, HQ585/80, HQ690/60; Chroma Technology, Brattleboro, Vt.) to an intensified charge-coupled device (I-PentaMAX; Roper Scientific, Trenton, N.J.), which records adjacent images of a 120-×60-μm section of the surface in two colors.

Experimental Protocols

FRET-Based Method Using Nucleotide-Based Donor Fluorophore

In a first experiment, universal primer is hybridized to a primer attachment site present in support-bound chimeric polynucleotides. Next, a series of incorporation reactions are conducted in which a first nucleotide comprising a cyanine-3 donor fluorophore is incorporated into the primer as the first extended nucleotide. If all the chimeric sequences are the same, then a minimum of one labeled nucleotide must be added as the initial FRET donor because the template nucleotide immediately 3′ of the primer is the same on all chimeric polynucleotides. If different chimeric polynucleotides are used (i.e., the polynucleotide portion added to the bound oligonucleotides is different at least one location), then all four labeled dNTPs initially are cycled. The result is the addition of at least one donor fluorophore to each chimeric strand.

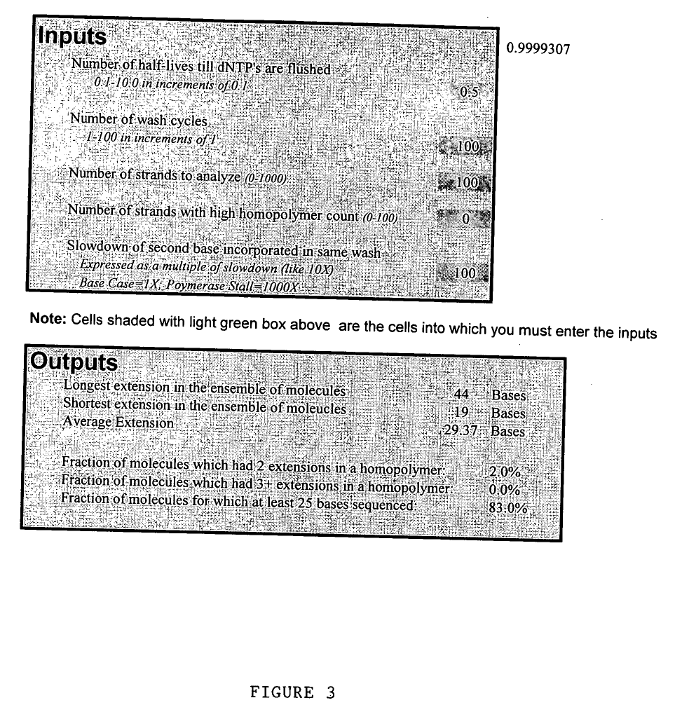

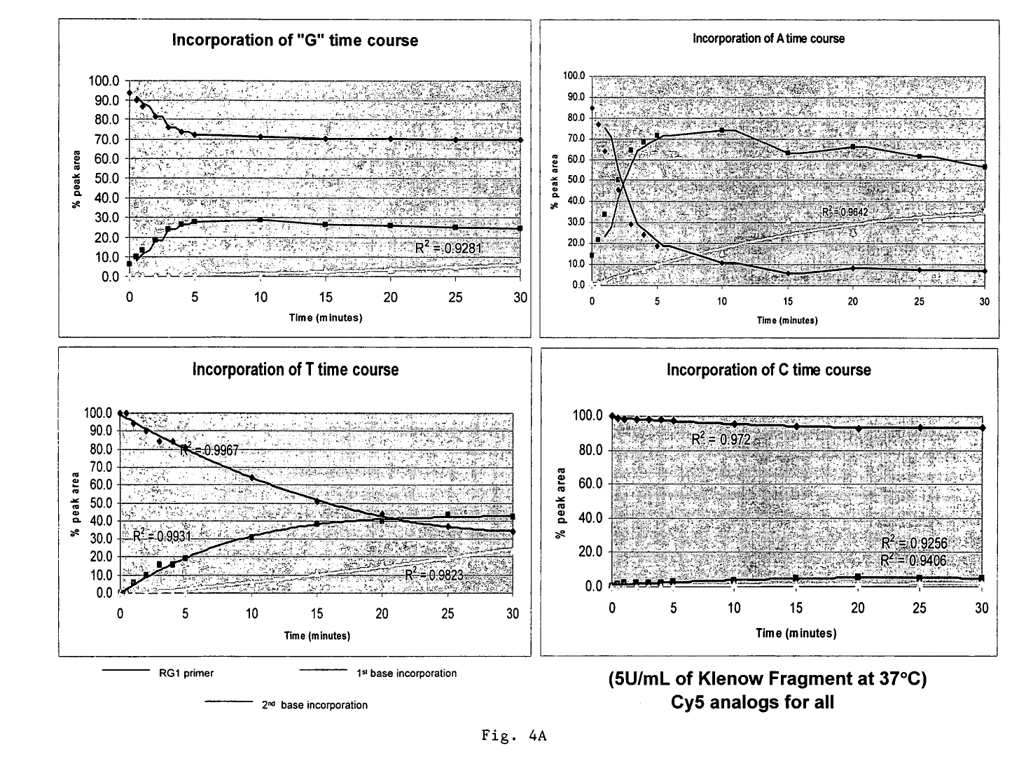

The number of initial incorporations containing the donor fluorophore is limited by either limiting the reaction time (i.e., the time of exposure to donor-labeled nucleotides), by polymerase stalling, or both in combination. The inventors have shown that base-addition reactions are regulated by controlling reaction conditions. For example, incorporations can be limited to 1 or 2 at a time by causing polymerase to stall after the addition of a first base. One way in which this is accomplished is by attaching a dye to the first added base that either chemically or sterically interferes with the efficiency of incorporation of a second base. A computer model is constructed using Visual Basic (v. 6.0, Microsoft Corp.) that replicates the stochastic addition of bases in template-dependent nucleic acid synthesis. The model utilizes several variables that are thought to be the most significant factors affecting the rate of base addition. The number of ½ lives until dNTPs are flushed is a measure of the amount of time that a template-dependent system is exposed to dNTPs in solution. The more rapidly dNTPs are removed from the template, the lower will be the incorporation rate. The number of wash cycles does not affect incorporation in any given cycle, but affects the number bases ultimately added to the extending primer. The number of strands to be analyzed is a variable of significance when there is not an excess of dNTPs in the reaction. Finally, the slowdown rate is an approximation of the extent of base addition inhibition, usually due to polymerase stalling. The homopolymer count within any strand can be ignored for purposes of this application. FIG. 3 is a screenshot showing the inputs used in the model.

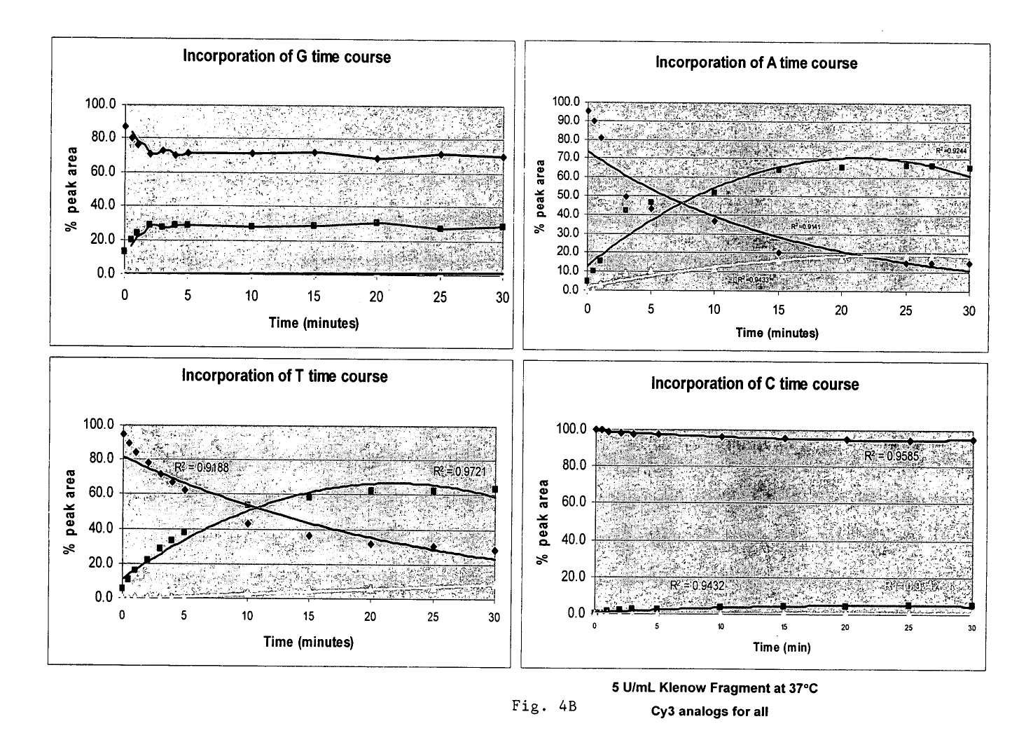

The model demonstrates that, by controlling reaction conditions, one can precisely control the number of bases that are added to an extending primer in any given cycle of incorporation. For example, as shown in FIG. 4, at a constant rate of inhibition of second base incorporation (i.e., the inhibitory effect of incorporation of a second base given the presence of a first base), the amount of time that dNTPs are exposed to template in the presence of polymerase determines the number of bases that are statistically likely to be incorporated in any given cycle (a cycle being defined as one round of exposure of template to dNTPs and washing of unbound dNTP from the reaction mixture). As shown in FIG. 4A, when time of exposure to dNTPs is limited, the statistical likelihood of incorporation of more than two bases is essentially zero, and the likelihood of incorporation of two bases in a row in the same cycle is very low. If the time of exposure is increased, the likelihood of incorporation of multiple bases in any given cycle is much higher. At a constant rate of polymerase inhibition (assuming that complete stalling is avoided), the time of exposure of a template to dNTPs for incorporation is a significant factor in determining the number of bases that will be incorporated in succession in any cycle. Similarly, if time of exposure is held constant, the amount of polymerase stalling will have a predominant effect on the number of successive bases that are incorporated in any given cycle (See, FIG. 4B). Thus, it is possible at any point in the sequencing process to add or renew donor fluorophore by simply limiting the statistical likelihood of incorporation of more than one base in a cycle in which the donor fluorophore is added.

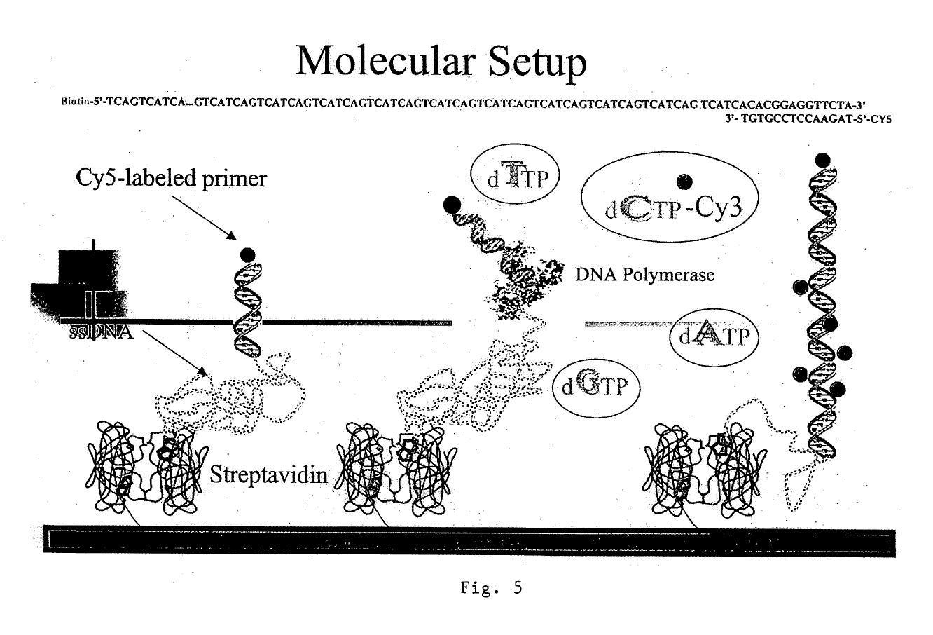

Upon introduction of a donor fluorophore into the extending primer sequence, further nucleotides comprising acceptor fluorophores (here, cyanine-5) are added in a template-dependent manner. It is known that the Foster radius of Cy-3/Cy5 fluorophore pairs is about 5 nm (or about 15 nucleotides, on average). Thus, donor must be refreshed about every 15 bases. This is accomplished under the parameters outlined above. In general, each cycle preferably is regulated to allow incorporation of 1 or 2, but never 3 bases. So, refreshing the donor means simply the addition of all four possible nucleotides in a mixed-sequence population using the donor fluorophore instead of the acceptor fluorophore every approximately 15 bases (or cycles). FIG. 5 shows schematically the process of FRET-based, template-dependent nucleotide addition as described in this example.

The methods described above are alternatively conducted with the FRET donor attached to the polymerase molecule. In that embodiment, donor follows the extending primer as new nucleotides bearing acceptor fluorophores are added. Thus, there typically is no requirement to refresh the donor. In another embodiment, the same methods are carried out using a nucleotide binding protein (e.g., DNA binding protein) as the carrier of a donor fluorophore. In that embodiment, the DNA binding protein is spaced at intervals (e.g., about 5 nm or less) to allow FRET. Thus, there are many alternatives for using FRET to conduct single molecule sequencing using the devices and methods taught in the application. However, it is not required that FRET be used as the detection method. Rather, because of the intensities of the FRET signal with respect to background, FRET is an alternative for use when background radiation is relatively high.

Non-FRET Based Methods

Methods for detecting single molecule incorporation without FRET are also conducted. In this embodiment, incorporated nucleotides are detected by virtue of their optical emissions after sample washing. Primers are hybridized to the primer attachment site of bound chimeric polynucleotides. Reactions are conducted in a solution comprising Klenow fragment Exo-minus polymerase (New England Biolabs) at 10 nM (100 units/ml) and a labeled nucleotide triphosphate in EcoPol reaction buffer (New England Biolabs). Sequencing reactions takes place in a stepwise fashion. First, 0.2 μM dUTP-Cy3 and polymerase are introduced to support-bound chimeric polynucleotides, incubated for 6 to 15 minutes, and washed out. Images of the surface are then analyzed for primer-incorporated U-Cy5. Typically, eight exposures of 0.5 seconds each are taken in each field of view in order to compensate for possible intermittency (e.g., blinking) in fluorophore emission. Software is employed to analyze the locations and intensities of fluorescence objects in the intensified charge-coupled device pictures. Fluorescent images acquired in the WinView32 interface (Roper Scientific, Princeton, N.J.) are analyzed using ImagePro Plus software (Media Cybernetics, Silver Springs, Md.). Essentially, the software is programmed to perform spot-finding in a predefined image field using user-defined size and intensity filters. The program then assigns grid coordinates to each identified spot, and normalizes the intensity of spot fluorescence with respect to background across multiple image frames. From those data, specific incorporated nucleotides are identified. Generally, the type of image analysis software employed to analyze fluorescent images is immaterial as long as it is capable of being programmed to discriminate a desired signal over background. The programming of commercial software packages for specific image analysis tasks is known to those of ordinary skill in the art. If U-Cy5 is not incorporated, the substrate is washed, and the process is repeated with dGTP-Cy5, dATP-Cy5, and dCTP-Cy5 until incorporation is observed. The label attached to any incorporated nucleotide is neutralized, and the process is repeated. To reduce bleaching of the fluorescence dyes, an oxygen scavenging system can be used during all green illumination periods, with the exception of the bleaching of the primer tag.

In order to determine a template sequence, the above protocol is performed sequentially in the presence of a single species of labeled dATP, dGTP, dCTP or dUTP. By so doing, a first sequence can be compiled that is based upon the sequential incorporation of the nucleotides into the extended primer. The first compiled sequence is representative of the complement of the chimeric polynucleotide. As such, the sequence of the chimeric polynucleotides can be easily determined by compiling a second sequence that is complementary to the first sequence. Because the sequence of the oligonucleotide is known, those nucleotides can be excluded from the second sequence to produce a resultant sequence that is representative of the target nucleic acid.

1. A substrate for use in sequencing nucleic acids, the substrate comprising:
a solid support; and
a plurality of oligonucleotides, each having the same sequence, attached to said solid support in a spatial arrangement such that each of said oligonucleotides is individually optically resolvable,
wherein each of said oligonucleotides comprises
at least five nucleotides;
a primer attachment site; and
a terminal attachment site for attaching a target polynucleotide.

2. The substrate of claim 1, wherein each of said oligonucleotides comprises between about 7 nucleotides and about 100 nucleotides.

3. The substrate of claim 1, further comprising a plurality of target polynucleotides, each being attached to said terminal attachment site of a different one of said oligonucleotides.

4. The substrate of claim 1, further comprising a plurality of primers, each having the same sequence and being capable of hybridizing to said oligonucleotides.

5. The substrate of claim 1, wherein each of said oligonucleotides is attached to said solid support via a linker.

6. The substrate of claim 5, wherein said linker is a biotin/avidin couple.

7. The substrate of claim 5, wherein said linker is digoxigenin/anti-digoxigenin.

8. The substrate of claim 3, wherein said substrate comprises between about 50 and about 100,000 target polynucleotides, each being attached to said terminal attachment site of a different one of said oligonucleotides.

9. A kit comprising the substrate of claim 4 and a polymerase enzyme capable of adding nucleotides to said primers in a template-dependent manner.

10. The substrate of claim 3, wherein each of said target polynucleotides is attached to said terminal attachment site of a different one of said oligonucleotides through blunt-end or cohesive-end ligation.

11. A method for sequencing a target nucleic acid, the method comprising:
exposing the substrate of claim 3 to a plurality of primers, each having the same sequence and capable of hybridizing to said oligonucleotides;
extending said primer in the presence of one or more nucleotides comprising a detectable label; and
detecting label incorporated into said extended primer, thereby to determine the sequences of said target nucleic acids.

12. A method for sequencing nucleic acids, the method comprising:
attaching a plurality of oligonucleotides, each having the same sequence, to a surface of a solid support in a spatial arrangement such that each of said oligonucleotides is individually optically resolvable,
attaching each of a plurality of target polynucleotides to a different one of said oligonucleotides, producing a plurality of chimeric polynucleotides;
exposing said chimeric polynucleotides to a primer capable of hybridizing to said oligonucleotides;
extending said primer in the presence of one of more nucleotides comprising a detectable label; and
detecting label incorporated into said extended primer, thereby to determine the sequences of said target nucleic acids.

13. The method of claim 12, wherein said extending step comprises extending said primer in the presence of a single species of labeled nucleotide and said detecting step comprises detecting said labeled nucleotide if it is incorporated into said extended primer.

14. The method of claim 13, further comprising repeating said extending and detecting steps sequentially.

15. The method of claim 13, wherein said single species of labeled nucleotide is selected from the group consisting of dUTP, dATP, dCTP and dGTP.

16. The method claim 12, wherein said label is an optically-detectable label.

17. The method of claim 16, wherein said optically-detectable label is a fluorescent label.

18. The of claim 17, wherein said fluorescent label is selected from the group consisting of a fluorescein, a rhodamine, a phosphor, a polymethadine dye derivative, a fluorescent phosphoramidite, a texas red dye, a green fluorescent protein, an acridine, a cyanine, a cyanine 5 dye, a cyanine 3 dye, a 5-(2′-aminoethyl)-aminonaphthalene-1-sulfonic acid (EDANS), a BODIPY, an ALEXA, and a derivative or modification of any of the foregoing.

19. The method of claim 12, wherein said step of attaching each of a plurality of target nucleic acids occurs prior to said step of attaching a plurality of oligonucleotides.

20. The method of claim 12, wherein said providing step comprises
attaching said oligonucleotides to said surface of said solid support; and
attaching each of a plurality of target polynucleotides to a different one of said oligonucleotides.

21. The method of claim 20, wherein said step of attaching each of said plurality of target polynucleotides occurs prior to said step of attaching said oligonucleotides.

22. The method of claim 12, wherein said step of attaching each of said plurality of target nucleic acids comprises blunt-end or cohesive-end ligation.

23. The method of claim 12, further comprising the step of compiling a sequence of a complement of each of said target nucleic acids based upon sequential incorporation of said nucleotides into said extended primer.
